(19) United States
(12) Patent Application Publication
(10) Pub. No.: US 2022/0415496 A1
(43) Pub. Date: Dec. 29, 2022

(54) PET HEALTH MONITORING SYSTEM

(71) Applicant: Full Life Wellness, LLC, Phoenix, AZ (US)

(72) Inventors: William Levy, Fountain Hills, AZ (US); Scott Eller, Phoenix, AZ (US)

(73) Assignee: Full Life Wellness, LLC, Phoenix, AZ (US)

(21) Appl. No.: 17/900,911

(22) Filed: Sep. 1, 2022

Related U.S. Application Data
(63) Continuation-in-part of application No. 17/568,548, filed on Jan. 4, 2022.
(60) Provisional application No. 63/133,583, filed on Jan. 4, 2021.

(51) Int. Cl. G16H 40/63

(52) U.S. Cl. CPC G16H 40/63

(57) ABSTRACT

A system enables monitoring and improving one or more of the habits, diet, exercise, and activities related to a pet's health and includes a network having a food supplier and a food preparation device connected to the network and arranged to enable specific food type and quantity to be supplied to the pet's parent automatically according to usage.

### FIELD

The present invention is related to pet parenting. More specifically, the invention is related to a system for monitoring and improving the habits, diet, exercise, activities, and other facets of a pet's health.

### BACKGROUND

A common and age-old problem is that pets are prone to overeat, live an inactive lifestyle, and do other things which may shorten their already too short lives. Without an incentive to do otherwise, pet's parents are prone to overfeed and pamper their pets, which adds to that shortening.

There is the need, and it may be an objective of the invention, to provide a system to improve the habits, diet, exercise, activities, and other facets of a pet's health.

There also is the need, and it may be an objective of the invention, to provide such a system including a food preparation device capable of providing a healthful amount of food or a healthful type.

There also is the need, and it may be an objective of the invention, to provide such a device which is connectable to a network for remote assistance.

There also is the need, and it may be an objective of the invention, to provide such a network which is wireless.

There also is the need, and it may be an objective of the invention, to provide such a system which includes a smartphone app capable of communicating through the network and adapted to receive input from the pet's parent and to provide a meal plan and/or health plan according thereto to the pet's parent.

There also is the need, and it may be an objective of the invention, to provide such a smartphone app capable of providing feedback from the device to the network.

Further needs and objectives will become apparent upon perusal of the disclosure herein.

### SUMMARY

The herein disclosed exemplary system, including the herein disclosed exemplary Device, combines the ability to monitor and improve the habits, diet, exercise, activities, and other facets of a pet's health, with the ability to allow the pet's parent to control the same according to a dietary plan based on the pet's specific needs.

The device may be capable of preparing the pet a healthful amount of food or a healthful type, according to a calculated meal plan. The device may be connectable to a network for data capture and exchange. The network may be wireless.

The system may include a website and/or a smartphone app capable of communicating through the network and adapted to receive pet-specific information from the pet's parent, process that information into a health plan including a meal plan, and to provide meals according thereto to the pet's parent for preparing through the device. The website and/or smartphone app may be capable of providing feedback from the device to the pet's parent.

Further features of the invention will become apparent upon perusal of the disclosure herein.

### BRIEF DESCRIPTION OF THE DRAWINGS

Exemplary embodiments of systems for monitoring and improving the habits, diet, exercise, activities, and other facets of a pet's health in accordance with or useful in practicing the invention is shown in the accompanying Drawings, of which;

FIG. 1 is a schematic view of a first system;

FIG. 2 is a view of the food preparation device of the system of FIG. 1 being loaded with food cups;

FIG. 3 is a view of the food preparation device of the system of FIG. 1 in its loaded and ready state;

FIG. 4 is a cross-sectional view of the food preparation device of the systems of FIG. 1 in its loaded and ready state;

FIG. 5 is a view showing the underside of the food cups of FIG. 2;

FIG. 6 is a flow diagram of the operation of the system of FIG. 1; and

FIG. 7 is a flow diagram of the operation of a second system.

### DESCRIPTION OF EXEMPLARY EMBODIMENTS

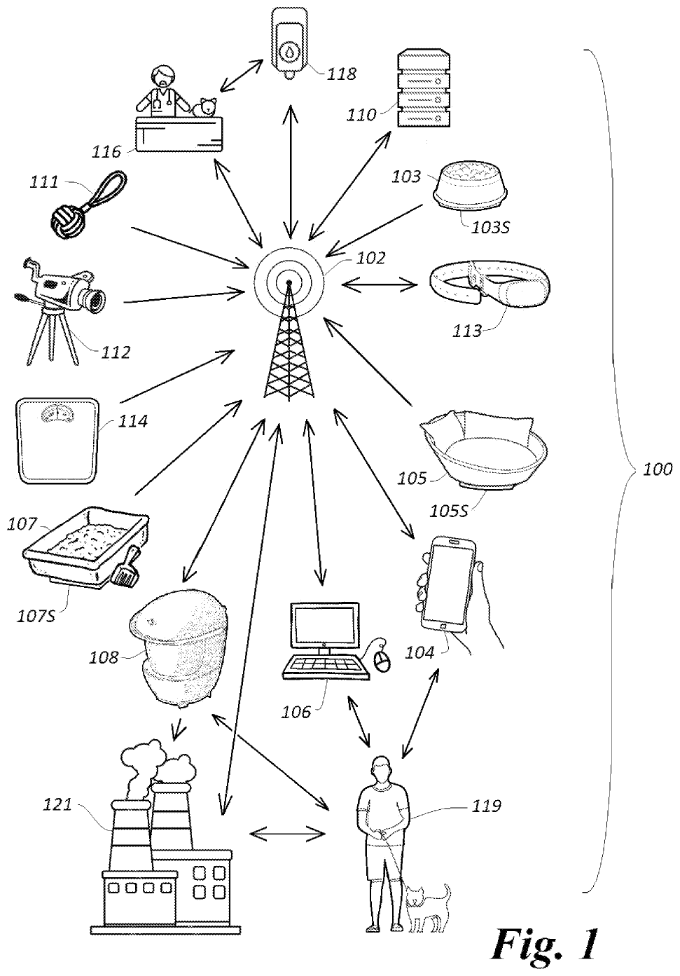

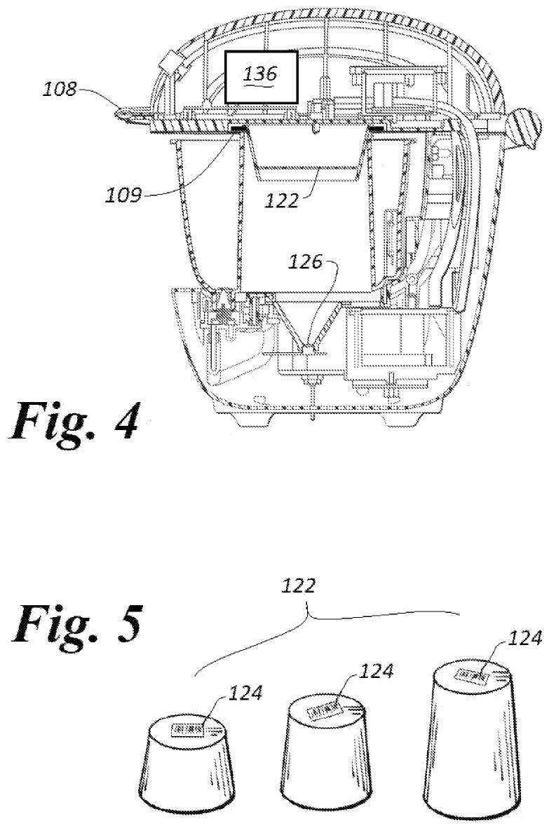

Referring first to FIGS. 1 through 6, a first exemplary system 100 for monitoring and improving the habits, diet, exercise, activities, and other facets of a pet's health is shown. The system includes some or all of the following components; a wireless or Internet network 102, a food/water/treat serving bowl 103, a smartphone 104, a pet bed 105, a personal computer 106, a litter box 107, a food preparation device 108, a server 110, one or more pet toys 111, an activity monitor 112, a remote pet collar 113, a pet scale 114, veterinarian service 116, a medicine dispenser 118, a pet parent 119, and a pet food supplier 121.

While FIG. 1 shows an icon of a wireless antenna to symbolize the network, the icon may also be interpreted to represent a hardwired Internet network or such. And while the activity monitor 112 is shown generically as a camera icon, and it may be as simple as a security camera which watches the pet and either records or provides a live stream of its activities to be viewed by the pet's parent or the veterinarian service either real time or recorded, it can additionally or alternatively be any one or more of a variety of things and serve any one or more of a variety of functions that monitor the pet's activities and report those to the network.

Regarding the food preparation device, this application claims benefit to co-invented and co-owned U.S. Pat. No. 10,966,409 issued on 6 Apr. 2021, and to co-pending, co-invented, and co-owned U.S. application Ser. No. 17/150,758 filed on 15 Jan. 2021, Ser. No. 16/175,145 filed on 30 Oct. 2018, and Ser. No. 17/568,548 filed on 4 Jan. 2022. The descriptions of the food preparation devices therein are incorporated herein in their entireties by reference and are not repeated herein for brevity, but any reference made herein to a food preparation device should be considered fully disclosed by such incorporation by reference excepting that the version of the device of the present application is equipped with a networking interface 136 for use in the present system. And while the icon of the food preparation device in FIG. 1 shows the food preparation device of U.S. Pat. No. 10,966,409, it should be understood that any pet food preparation device capable fulfilling the role required by the system could be substituted therefore.

A key aspect of the system is that the food is provided by a single supplier in capsules certifiable by the food preparation device for content and weight so that the diet of the pet can be carefully monitored and controlled, and the pet parent's inventory of supplies can be automatically and passively refreshed in response to use and depletion. Additionally, the food preparation device will not only refuse to operate absent a meal provided by that provider and according to the approved meal plan but may also report the attempt to use an unapproved capsule to the network, such as to void any guarantee of results that may be offered by the supplier or the manager of the system.

The supplier can not only use the meal consumption information to retard counterfeits but can also monitor the pet parent's food inventory so that a subscription arrangement can ensure that replacement meals according to the meal plan are timely and conveniently provided without direct management by the pet parent.

The same or a different supplier, such as the veterinarian service, may monitor medicine consumption and inventory and employ an additional subscription arrangement to ensure that replacement medications according to of the meal plan are timely and conveniently provided without direct management by the pet parent.

The smartphone 104 is carried by the pet's parent and loaded with a smartphone app 128 adapted to both receive and transmit information about the pet, its activities, its food consumption, and its care through the network.

The optional personal computer (PC) 106 may also be loaded with the same app, a more PC-specific program, or may access the system through a system website via the Internet.

The server 110 may be located where convenient and practical, such as at a server hosting facility or at a system managing headquarters. The server is ultimately controlled and operated by a system manager or its assigns, who may simultaneously employ the server to manage many equivalent pet health monitoring and improving systems.

The pet scale 114 is capable of accurately weighing the pet, and is equipped with network access capabilities, either through the food preparation device or in parallel therewith, so that it can communicate wirelessly or through the Internet to one or more of the other components of the system. The scale thereby transmits the recorded weight to the network.

The bowl 103 is equipped with a scale 103S to enable the measurement and reporting of exactly how much of the food the pet has actually consumed to monitor the pet's adherence to the health plan.

The pet bed 105 is equipped with a scale 105S to enable the confirmation and reporting of the time and timing spent on the bed by the pet to monitor the pet's adherence to the health plan. This scale may serve as and substitute for the pet scale 114.

The litterbox 107 is equipped with a scale 107S to enable the confirmation and reporting of the frequency and amount of pet defecation. This scale may also serve as and substitute for the pet scale 114.

The toy 111 is equipped with a motion detector to enable the confirmation and reporting of the frequency and amount of pet play.

The medication dispenser 118 is of the type that releases a preloaded medication or vitamin dosage so that it becomes available for the pet's consumption. This dispenser also has the ability for network access, either through the food preparation device or in parallel therewith, so that it can communicate wirelessly or through the Internet to one or more of the other components of the system. The medication dispenser thereby reports the dispensed medication to the network.

The veterinarian service 116 may be an actual private veterinarian of the parent's choosing who has access and capability to use the system or it may be a veterinarian service provided by the system manager, or it may be an autonomous virtual veterinarian.

An additional and more sophisticated monitoring system subsystem may be realized by remote pet collar 113 connected to the network by Bluetooth or such. The collar may include a geolocation (GPS) system for tracking the pet's activities and movements to assist in gauging such things as the amount of distance travelled, or the amount/quality/timing of rest.

The collar might include features such as a GPS tracker, a pace monitor, a heartbeat monitor, a thermometer, and/or means of measuring and reporting numerous other aspects of the pet's health and activities that can be transmitted to the network, either through the food preparation device or independently, such that they are available to the parent, veterinarian service, app and system. The collar may be adapted to receive commands from the network upon which corrective signals are given to the pet when undesirable behavior recognized, such as roaming out of a given area, excessive barking, or to wake the pet when excessive inactivity is determined.

The activity monitor(s) may also be or include a microphone or such to monitor for crying or barking, which can be indicators of stress and agitation.

The information is transmitted, by all components or some, through the food preparation device or directly to the server via the wireless or Internet connection, and all information placed onto the network by any of the components is available for use and viewing by any other of the other components which may benefit from the information.

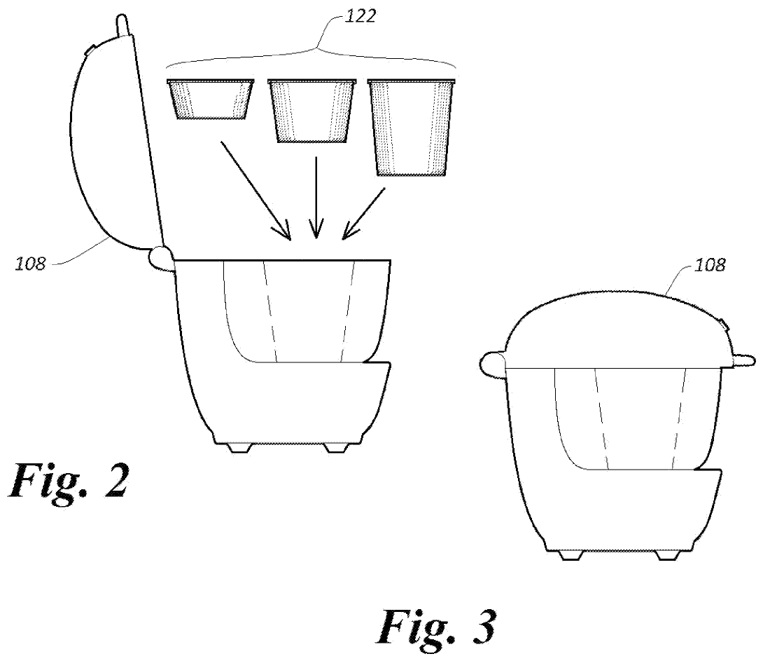

As explained in patent Ser. No. 10/966,409 and as depicted in FIGS. 2, 4, and 5, the food preparation device may be adapted to receive, identify, and certify cups of food 122, . . . each having a barcode 124 on its underside which is read by a scanner 126 in the food preparation device and used to identify and verify the volume and ingredients of the food they contain. These food cups are available from the system manager or its assigns in a variety of sizes and with a variety of food ingredients. The food ingredients may be formulated according to pet age, breed, health, weight and/or dietary needs or restrictions that had been provided by the parent, the veterinarian service and/or the other components of the system. The food scale 109 is adapted to measure the weight of food in the container to further confirm that it is indeed full of the expected amount and not, for instance, a re-used container filled with something else or something less.

Once the parent has set up a subscription with the system manager, received the feeding machine from the system manager or one of its assigns, loaded the app into a smartphone or through the PC, and reported the pet-specific information, and had then received the health plan, including at least a meal plan, and the pet specific foods indicated thereby, the parent loads the upcoming meal into the food preparation device. The device may automatically prepare the meal and/or may alert the parent by alarm and/or notification through the app, at the times that meals should be prepared and served.

The app will automatically track and monitor not only the meal provided to the pet but also the pet's consumption of all or part of that meal through the scale 103S of the food bowl 103, if provided, and will transmit that through the network.

The other devices may enable real time monitoring or may alert the parent by alarm and/or notification through the app, as their function should be used or activated according to the remainder of the health plan.

The app may cause the activity monitor(s) to record and transmit the desired activities and health readings and transmit them to the network.

The various components of the network will create information from such things as food consumption, medicine usage, pet weight, litter weight, and activities.

The veterinarian service, if employed, or an algorithm within the server or the app, will use that information to adjust or to advise adjustment of related portions of the health plan, including the meal plan, as needed, and will transmit that to the network and to at least the app or website. Or the server may determine the adjustments and proper advice through an algorithm programed therein and employ it to adjust the health plan, including at least the meal plan, as needed, and will transmit that to the network and to at least the app or website.

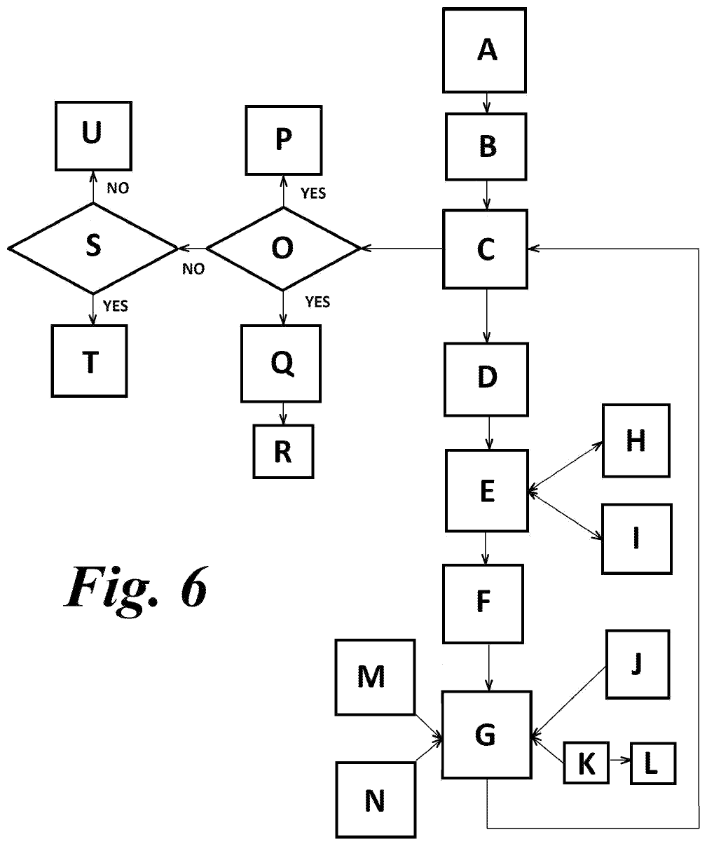

Referring to FIG. 6, there is shown a flow chart for the system, in which each block is identified by a letter corresponding to the following list;
A. After having purchased a subscription from the system manager and downloaded the app or created an Internet account, the pet's parent inputs the pet's breed, weight, age, health history, and usual activity level information into the smartphone app or website.
B. The app or website communicates that information to an algorithm in the server.
C. The algorithm calculates the pet's caloric need, nutritional need, medicinal need, and activity need based on the information and makes a daily plan recommendation to the parent, including at least a meal plan recommendation.
D. Once the meal plan recommendation is accepted by the parent, the food preparation device, meals, and other devices (if employed) are sent to the parent.
E. The food preparation device is connected to a wi-fi network via Bluetooth and a smart phone app onboarding method, or is connected via the Internet through the PC. The other devices (if employed) are connected either directly or through the food preparation device and activated. The device may issue a “Feed Pet Now” alert when appropriate, through the app and/or by alarm.
F. The food preparation device's bar code reader recognizes each meal inserted into the food preparation device via the bar code on the bottom of the meal cup and begins the appropriate preparation cycle.
G. The food preparation device communicates identification and preparation of the meal to the network, and the veterinarian service or server's algorithm uses that to monitor and adjust caloric and nutritional intake, and to produce a maintenance report which it transmits to the network, including to at least the app and/or the website.
H. The pet scale (if employed) is connected to the network either directly or through the food preparation device and communicates things like the pet's consumption, weight, and activities to the network, and the veterinarian service or the server's algorithm uses that to monitor and adjust diet, recommended activities, etc., and to produce the maintenance report and an adjusted health plan.
I. The activity monitors (if employed), and other components (if employed), are connected to the network either directly or through the food preparation device and communicate the pet's activities to the network, and the veterinarian service or the server's algorithm uses that to monitor and adjust things like caloric intake, recommended activities, and medication, and to produce the maintenance report and an adjusted health plan. The activity monitor may issue an “Exercise Pet Now” alert when appropriate, through the app and/or by alarm.
J. The food preparation device may issue a “Feed Pet Now” alert when appropriate, through the app and/or by alarm.
K. The pet scale may issue a “Weigh Pet Now” alert when appropriate, through the app and/or by alarm.
L. The alerts may be issued via email, text, or smart phone app notifications and/or by sound/visual alarms by the applicable devices.
M. Is there a need to adjust caloric intake?
N. New or existing daily plan, weight target, and/or recommended activities may be formulated by the veterinarian service (if employed).
O. The new or existing daily plan, weight target, and/or recommended activities may be uploaded by the veterinarian service (if employed) to a tele-health portal.
P. The tele-health portal may make the new or existing daily plan, weight target, and/or recommended activities available to the parent via the app or website.
Q. Are the food preparation device, scale, and/or activity monitor functioning properly?
R. A new food preparation device, scale, and/or activity monitor is sent to the parent, or the parent is instructed to purchase/order one.
S. The parent keeps using the existing food preparation device, scale, and/or activity monitor.

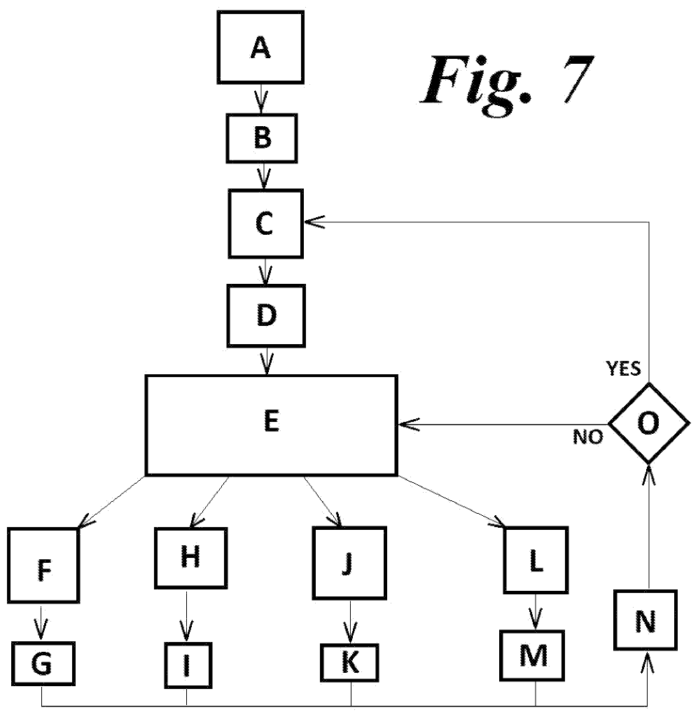

Referring to FIG. 7, there is shown a flow chart for an alternate system, in which each block is identified by a letter corresponding to the following list;
A. After having purchased a subscription from the system manager and downloaded the app or created an Internet account, the pet's parent inputs the pet's breed, weight, age, health history, and usual activity level information into the smartphone app or website.
B. The app or website communicates that information to an algorithm in the server.
C. The algorithm calculates the pet's health plan, including at least a meal plan, based on the information and makes a plan recommendation to the parent, including at least a meal plan recommendation.
D. The server communicates the health plan to the network.
E. The app issues alerts to the parent to remind of feeding, medication, health monitoring and exercise, as due. The app provides a report received from the algorithm and including at least pet feeding history, health history, weight history, and medication history on demand through the app and/or website.
F. The food preparation device issues a “Feed Now” alert through the app and/or by the device.
G. The food preparation device identifies the meal inserted and reports that to the network.
H. The scale (if employed) issues a “Weigh Now” alert through the app and/or by the scale or food preparation device.
I. The parent weighs the pet on the scale (if employed) and the scale reports the measured weight to the network.
J. The activity monitors (if employed) issue a “Monitor Now” alert through the app and/or the monitor or the device.
K. The parent measures recommended health conditions of the pet using the appropriate activity monitor (if employed) and the monitor reports those to the network.
L. The medication dispenser (if employed) issues a “Medicate Now” alert through the app and/or the dispenser or the food preparation device.
M. The medication dispenser dispenses and reports to the network.
N. All events, results, and information are communicated wirelessly or via the Internet to the server's algorithm.
O. In violation of the health plan?

It is anticipated that some of the steps and functions of each of the two systems above may be removed or may be added from one to the other.

While the forgoing provides exemplary embodiments are meant to demonstrate key aspects and advantages of the invention, it should be understood that the invention is not meant to be limited thereto, and the invention should be limited only by the following claims, including all equivalents thereto.

We claim:

1. A system for monitoring and improving one or more of the habits, diet, exercise, and other activities related to a pet's health comprising:
a network, a pet parent, a pet food supplier, and a food preparation device;
wherein the pet food supplier supplies pet food to the pet parent for placement into the food preparation device;
wherein the network is configured to enable communication between the pet parent, the pet food supplier, and the food preparation device;
and wherein the food preparation device is adapted to confirm that pet food placed therein is derived from the pet food supplier and to communicate receipt of the confirmed pet food with one or both of the pet parent and the pet food supplier such that an adequate pet parent inventory of pet food is supplied and maintained.

2. The system of claim 1 further comprising a smartphone having an app therewithin to enable the communication between the pet parent, the pet food supplier, and the food preparation device.

3. The system of claim 2 wherein the food preparation device is adapted to confirm and communicate the contents and weight of the confirmed petfood.

4. The system of claim 3 wherein the pet food is supplied in prepackaged food capsules having food sealed therewithin and an identifier, and the food preparation device has a reader adapted to read the identifier.

5. The system of claim 4 wherein the identifier contains pet food content information.

6. The system of claim 5 wherein pet food content information includes calories and weight of the pet food within the prepackaged food capsules.

7. The system of claim 6 wherein the food preparation device is adapted to prepare meals from the prepackaged food capsules.

8. The system of claim 7 wherein the food preparation device is adapted to receive prepackaged food capsules of various volumes such that the meal code of each is visible to the meal code reader regardless thereof.

9. The system of claim 8 further comprising means for calculating a health plan from pet-specific information.

10. The system of claim 9 wherein the pet specific information received includes one or more data of the pet's breed, age, weight history, diet history, activity history, medication history, and health history.

11. The system of claim 10 wherein the health plan includes a pet-specific meal plan.

12. The system of claim 11 wherein the pet food supplied by the pet food supplier is according to the pet-specific meal plan.

13. The system of claim 12 further comprising one or more items from the group including a serving bowl, a pet bed, a personal computer, a litter box, a pet toy, an activity monitor, a remote pet collar, and a pet scale, and wherein the network further enables communication between the pet parent, the pet food supplier, the food preparation device, and the one or more.

14. The system of claim 13 wherein the health plan further includes a pet-specific activity plan.

15. The system of claim 14 wherein the pet-specific activity plan is derived from the pet-specific information.

16. The system of claim 15 further comprising a veterinarian to create the health plan.

17. The system of claim 16 wherein the veterinarian creates the health plan in consideration of the pet specific information.

18. The system of claim 17 wherein the health plan is amended as-needed according to information received from the one or more items.

19. A system for monitoring and improving one or more of the habits, diet, exercise, and other activities related to a pet's health comprising:
a network enabling wireless and/or wired communication;
one or both of an algorithm programmed into a server in communication with the network and a veterinarian service connected to the network, to create a meal plan in accordance to received pet-specific data;
a food supplier in communication with the network and adapted to receive the meal plan and to provide food according thereto to a pet parent in sealed packages including an indicator of package contents;
a food preparation device adapted translate the indicator and confirm that its source is the food supplier and that its contents are accordance with the meal plan, and to either prepare the food for pet consumption when the indicator is translated to be from the food supplier and according to the meal plan or to refuse to prepare the food when the indicator is not translated to be from the food supplier or not according to the meal plan;
wherein the food preparation device is connected to the network and adapted to transmit food identification and preparation information thereto;
wherein the system communicates the food identification and preparation information to the food supplier to enable the supplier to monitor and automatically maintain the pet parent's food inventory and provide replacement food according to the meal plan.

20. The system of claim 19 further comprising one or both of pet weight and pet health monitors connected to the network and adapted to monitor one or both of pet ongoing weight and pet ongoing health and report one or both of those thereto, and wherein the one or both of the algorithm and the veterinarian service modifies the meal plan in accordance therewith.

21. The system of claim 20 wherein the food supplier modifies supply of food to the pet parent according to the modified meal plan.

22. The system of claim 21 further comprising one or both of a smartphone and a personal computer possessed by the pet parent, connected to the network, and enabled to inform the pet parent of pet weight progress and a pet feeding schedule.

23. The system of claim 22 wherein one or more of the food preparation device, the smartphone, and the personal computer are adapted to provide alerts to the pet parent in accordance with the pet feeding schedule.

24. The system of claim 19 wherein the one or both of the algorithm and the veterinarian service creates a health plan in accordance with the received pet-specific data;

25. The system of claim 24 further comprising one or both of pet weight and health monitors connected to the network and adapted to monitor one or both of pet ongoing weight and pet ongoing health and report one or both of those thereto, and wherein the one or both of the algorithm and the veterinarian service modifies the meal plan in accordance therewith.

26. The system of claim 25 further comprising one or both of a smartphone and a personal computer possessed by the pet parent, connected to the network, and enabled to inform the pet parent of both pet health progress and pet feeding and activity schedules.

27. The system of claim 26 wherein one or more of the food preparation device, the smartphone, and the personal computer are adapted to provide alerts to the pet parent in accordance with the pet feeding and activity schedules.

28. The system of claim 27 wherein the pet-specific data includes one or more of the pet's breed, age, weight history, diet history, activity history, medication history, and health history.

29. The system of claim 28 wherein the pet ongoing health report includes one or more of the pet's recent food consumptions, the pet's recent activities, pet's recent medication, the pet's recent sleeping habits, the pet's recent defecation activity, and.
